(19) United States
(12) Patent Application Publication
(10) Pub. No.: US 2004/0101500 A1
(43) Pub. Date: May 27, 2004

(54) Method of preparation of an antiperspirant salt

(76) Inventors: Thomas Alexander Ashcroft, Merseyside (GB); Johannes Breker, Ludwigshafen (DE); Keith Gosling, Cheshire (GB); Bruno Kaufmann, Ludwigshafen (DE); Margaret Philippa Smith, Merseyside (GB)

Correspondence Address: VENABLE, BAETJER, HOWARD AND CIVILETTI, LLP, P.O. BOX 34385, WASHINGTON, DC, 20043-9998, US

(21) Appl. No.: 10/276,589

(22) PCT Filed: Apr. 27, 2001
(86) PCT No.: PCT/EP01/04766

(30) Foreign Application Priority Data
May 19, 2000 (GB) 0012267.1

(51) Int. Cl. A61K 7/06

(52) U.S. Cl. 424/70.1

(57) ABSTRACT

A method of providing a salt having antiperspirant activity and having an empirical formula Al₂(OH)₆₋ₐXₐ.YH₂O wherein X is one of Cl, Br or I, Y ranges from 1.5 to 2.5, and a ranges from 0.8 to 1.33 includes heating an aqueous solution comprised of one of aluminum halide or HX, and aluminum metal for a time period sufficient for the aluminium metal to dissolve and provide a solution containing dissolved aluminum; and subsequently aging the solution containing dissolved aluminium for a time period ranging from 50 minutes to 21 days at a temperature ranging from 80 to 130° C. to provide a resulting solution having a ratio of Al to X ranging from 1.5:1 to 2.0:1 and having an aluminum concentration ranging from 0.5 to 3.8 wt %.

This invention relates to a method of preparation of a basic aluminium antiperspirant salt having enhanced activity, and to the resulting salts.

It is known to prepare an activated aluminium antiperspirant salt by dissolving aluminium metal in a heated aqueous solution of aluminium chloride. Typically, the aluminium is in the form of powder or pellets. The resultant aluminium compound is a polymeric basic aluminium halide salt, and has the empirical formula:

Al₂(OH)₆₋ₐXₐ.YH₂O

where a is typically from 0.7 to 3.0.

An example of such a preparation is to be found in “Ageing processes of Alumina Sol-Gels: Characterisation of New Aluminium Polyoxycations by ²⁷Al NMR Spectroscopy”, Fu and Nazar, Chem. Mater. 1991, 3, 602-610. In this teaching, a polymeric basic aluminium chloride salt is prepared at 60-95° C. purified, and then aged at 85° C. for various periods of time.

Other publications which generally disclose the preparation of aluminium polymers from a heated solution of aluminium chloride and aluminium in “single stepf” procedures, in a variety of methods and using different processing parameters, include U.S. Pat. No. 5,356,609, U.S. Pat. No. 5,358,694, EP 256,832, and EP 451,395.

Whilst the activated aluminium chlorhydrate (AACH) compounds made by prior methods have produced antiperspirant actives which have a generally relatively high efficacy in topical products, it is desirable that antiperspirant actives be made if possible of a higher efficacy. This is because topical products made utilising such actives can be made to have a higher efficacy than other commercially available products, thereby minimising the frequency and/or the intensity of wetness events witnessed by users.

A higher efficacy antiperspirant active is also desirable because it provides the opportunity to formulate products either of intermediate efficacy, or of an efficacy akin to that of currently available top efficacy commercial products, but in any event utilising a lesser amount of active in the topical product than is currently used. Such opportunities may provide for a possible cost reduction in the manufacture of the product, or reduced irritation in the topical product, since the concentration of the active material present is less than that in currently available commercial products. In addition, if the topical product contains a lesser amount of active, it may be easier to formulate, which in itself provides benefits.

As an associated aspect, we have found that a problem with antiperspirant actives for use in topical applications is that it is possible for their efficacy to decrease over a period of time. This clearly can present problems to the manufacturer, since it means not only that once an active salt has been made that it must be must be quickly used and formulated, but also that there is possibility that topical products manufactured with such salts may also find their efficacy decreases over a period of time.

We have surprisingly found after much experimental investigation that by selection of appropriate processing parameters, it is possible to provide an improved process for the production of high efficacy antiperspirant actives. The resulting novel antiperspirant actives have an efficacy which is generally at least equivalent to that provided by current production methods, and may also in certain embodiments be more stable than prior actives, providing sustained efficacy over a period of time.

Thus, according to a first aspect of the invention, there is provided a method of providing an antiperspirant active having the empirical formula:

Al₂(OH)₆₋ₐXₐ.YH₂O

wherein X is Cl, Br or I, Y represents an associated amount of water and is typically between 1.5 and 2.5, and a is 0.8 to 1.33, comprising heating an aqueous solution of aluminium halide or HX and aluminium metal for a time period sufficient for the aluminium metal to dissolve, and subsequently ageing the solution of dissolved aluminium for a period of 50 minutes—21 days at a temperature of 80-130° C., such that the Al/X ratio in the resulting solution is between 1.5:1 and 2.0:1, and such that the aluminium concentration in the resulting solution is between 0.5 and 3.8 wt %.

In the above aspect of the invention, the solution of dissolved aluminium used in the ageing step is that produced in the foregoing dissolution step, either following dilution with water or at its unadjusted concentration.

In a highly preferred embodiment, the resultant material is dried to provide a salt having a water content of less than 12 wt %.

Preferably, the halide ion is chloride.

In certain preferred embodiments, a is between 0.9 and 1.2, preferably between 1.0 and 1.15, and is most preferably 1.1. Conveniently, Y is between 1.5 and 2.5, more preferably between 1.7 and 2.3. In relation to the water content of the salts, the most convenient way of assessing this is with a moisture balance. It will however be appreciated that the values for water content given by this method do not bear a direct relationship to the values quoted in the empirical formula which are derived from elemental analyses. Measurements by moisture balance do however represent a practical and reproducible measure that relates to the activity and stability characteristics of the salts.

In an aspect of the method of the invention, the process may be divided into two distinct steps, both of which involve heating. The first involves the dissolution of aluminium metal in a solution of aluminium halide or HX such that the aluminium metal is fully dissolved. This dissolution is accompanied by evolution of hydrogen gas, hence, for practical reasons it is preferably and most conveniently carried out at temperatures less than or equal to 100° C., in particular 90-100° C., and especially 95-100° C. With appropriate equipment, capable of withstanding elevated pressure, higher temperatures may be used, however. The dissolution step at temperatures of 100° C. and concentrations described has typically been found to last approximately 4-9 hours.

When aluminium metal is used, it is highly desirable that high purity material (at least 99%, preferable at least 99.9%) is used. Trace metals (e.g. iron, cobalt, nickel and chromium) must be kept to a minimum to ensure that any antiperspirant product made from the active is suitable for cosmetic application.

The dissolution step may be carried out in a relatively concentrated solution, or may be carried out at a relatively dilute concentration, i.e. at or close to the same concentration as that at which the ageing step is carried out. The preferred concentration of aluminium in the ageing step is set out elsewhere in this application, and is dependent on other factors, in particular temperature at which the ageing step is carried out.

When the dissolution step is carried out at an aluminium concentration which is higher than that utilised during the ageing step; the dissolved aluminium containing solution is then diluted to the appropriate aluminium concentration prior to the ageing step.

It has however been found to be preferable that the dissolution step is carried out in relatively dilute form, i.e. with the aluminium concentration at or close to the concentration utilised during the ageing step. The aluminium antiperspirant salts resulting from carrying out the dissolution step at the relatively dilute concentration have been found to be slightly but significantly more active, in terms of their antiperspirant efficacy, than salts produced utilising a more concentrated aluminium dissolution step.

In addition, although not wishing to be bound by theory, it is thought significant that the resultant antiperspirant active salts have been built up through a process which has involved a build up in polymer size throughout, and has utilised a relatively dilute dissolution step. Thus, the process of the invention starts from aluminium halide or HX and elemental aluminium, which utilising a relatively dilute dissolution step come together to form relatively small particle size aluminium chlorhydrate polymers. These polymers are thought to increase in size during the ageing step to form the antiperspirant active salts according to the invention.

This is in contrast too many methods of producing activated aluminium chlorhydrate, which for example utilise commercially available aluminium chlorhydrate, which has a relatively large polymer size. Subsequent processes to age this material result in an activated aluminium chlorohydrate material which has smaller polymer size than the aluminium chlorohydrate from which is was produced, and hence have involved a depolymerisation step. Using the relatively concentrated dissolution step of the process of the invention, it is possible that the polymers so produced prior to the ageing step may have polymer sizes larger than those produced by the relatively dilute dissolution step, and thus also undergo depolymerisation during the ageing step to produce the antiperspirant active salt.

The subsequent ageing step, which conveniently can be carried out immediately successive to the dissolution step, is required to generate the appropriate polymer species in the solution to provide antiperspirant actives of suitable efficacy. In certain embodiments, it may be desirable to filter the reaction solution after the dissolution step, prior to the ageing step, in order to remove impurities in the solution.

It has also been found that the optimum concentration of aluminium polymers in the resulting solution may also be a function of the temperature at which the ageing step is carried out. Thus, for example, if the subsequent ageing step is carried out at 100° C., then conveniently the aluminium concentration in the resulting solution is in the range 1.4-2.1% by weight of aluminium, more preferably 1.6-1.95% by weight of aluminium. Where the ageing step is carried out at 120° C., conveniently the ageing step is carried out at an aluminium concentration of 2.3-2.7%, most preferably 2.42.6% by weight of aluminium. Where the ageing step is carried out at 130° C., conveniently the ageing step is carried out at an aluminium concentration of 3.2-3.8 wt %, most preferably 3.4-3.6% by weight of aluminium.

Conversely, the preferred ageing period is also dependent on the temperature at which the ageing step is conducted. For example, if the ageing step is carried out at 80° C., the ageing step may last for a period of up to 21 days, but preferably lasts for 3 to 15 days, more preferably 5 to 10 days, most preferably 6-8 days. If the ageing step is carried out at 100° C., preferably the ageing step is carried out for a period of 10 to 48 hours, more preferably 20 to 30 hours. If the ageing step is carried out at 120° C., the ageing step is preferably carried out for a period of 2 to 10 hours, more preferably 3 to 5 hours. If the ageing step is carried out at 130° C., the ageing step is preferably carried out for a period of 50 to 100 minutes, more preferably 70 to 80 minutes. In certain embodiments, preferably the ageing temperature is in the range 100-110° C.

In order to increase the overall rate at which the desired antiperspirant active is generated, it is preferable to perform the ageing step by sequential use of a high temperature followed a lower temperature. This procedure can also lead to a particularly desirable mixture of aluminium species and correspondingly good antiperspirancy performance for the salt produced. The procedure can beneficially be done using a higher concentration of aluminium in the high temperature ageing step than in the lower temperature ageing step. In a preferred procedure, the ageing step is performed at 110-130° C. for 0.5 to 10 hours, followed by a subsequent ageing step at 85-100° C. for 3 to 48 hours. It is generally found that the use of temperatures towards the top of these ranges enables good products to be formed after ageing times towards the bottom of the ranges indicated and vice-versa. It is further preferred that the aluminium concentration for the 110-130° C. stage is 2.5 to 3.5% by weight and that the aluminium concentration preferred for the 85-100° C. stage is 0.8 to 2.5% by weight.

Preferred aluminium solutions and salts according to the invention have a relatively low concentration of Al₁₃ species, as detected by ²⁷Al NMR techniques (vide infra), which species tend to be present in aluminium salts and solutions which have not been subjected to the appropriate ageing conditions. Conveniently, Al₁₃ species are present in aged solutions produced according to the invention at levels of less than 20 wt %, more preferably less than 10%, and often less than 7% by weight of the aluminium species.

We have also surprisingly found, somewhat contrary to certain prior art teachings, that small amounts of Band 0 polymers may be present in preferred salts and solutions made according to the invention. These Band 0 polymers, which are thought to be inactive as regards antiperspirant activity, and which have an effective diameter of 100 Angstroms or greater, may account for less than 10 wt %, more preferably less than 5 wt %, and often levels less than 2.0 wt % by weight of the aluminium species. In preferred salts and solutions they are often present at levels representing at least 0.1% by weight of the aluminium species. It is the presence of Band 0 polymers that accounts for cloudiness often observed in the solution in many of the prior art methods.

For the avoidance of doubt, it should be noted that in this specification, unless otherwise specified, quoted amounts of the various individual aluminium polymer species in the composition are expressed as percentages by weight of the aluminium polymers, and refer to the total aluminium species in the composition, including polymers in the so-called Band 0 range.

The combination of processing parameters described has been found to result in a process which can readily be carried out as a single step process, which provides an activated salt which is preferred, and which provides a particularly high degree of efficacy when incorporated into a topical antiperspirant composition.

In a further aspect, the invention also provides an antiperspirant active salt made according to the process of the invention.

Preferably, the dissolution step is carried out at temperatures of 90-100° C., more preferably 95-100° C. As is widely appreciated, increasing the temperature of a chemical reaction typically causes it to proceed faster, which is desirable from an economic stand point. Once the aluminium dissolution is complete, the ageing step can be carried out at higher temperatures (i.e. higher than 100° C.) in sealed vessels at elevated pressure. Such high temperatures enable shorter ageing periods and are therefore more desirable from an economic position.

Preferably, the ratio of Al/X in the final reaction solution (i.e. after the ageing step has been carried out, and before the dissolution step) is 1.7 or more, and may also preferably be 1.9 or less. More preferably it may be 1.75:1 or more, and may also be 1.85:1 or less; most preferably it is 1.8:1. All the Al/X ratios referred to in this specification are atomic ratios.

Conveniently, the water content in the resulting (dried) antiperspirant active salt is no lower than about 2%, is preferably at least about 4%, is more preferably at least 6%, and is even more preferably at least 8% by weight of the composition. Preferably, the water content of the salt is less than 12%, more preferably less than 10% by weight of the salt. These preferences are particularly relevant to antiperspirant active salts dried by spray drying.

It has been found that reducing the water content of the salt is desirable for the long term stability of the salt. With regard to the minimum water content, it is believed that whilst too little water is not in itself harmful to the stability of the salt, the drying regimes to which the salt needs to be subjected to get the water content to particularly low levels may be deleterious to the more antiperspirant active polymer species in the salt. Water content can conveniently be measured using a moisture balance. Water contents quoted in this application were measured using a Sartorius MA 30 moisture balance, on an “auto” programme with a set point of 100° C. Samples were stored in sealed vessels, introduced onto the balance at room temperature, and the temperature ramping programme started immediately. Quoted water content levels were based on an average value from a minimum of three repetitions.

Dried activated aluminium actives according to the invention can conveniently be isolated on an industrial scale by freeze drying or spray drying. Freeze drying is generally considered to be a less harsh drying technique, and hence may be considered to be a preferred drying method in certain circumstances, though spray drying may be considered to be a preferred technique in other circumstances, since it tends to result in a dried salt with a more consistent and desirable particle size distribution. This may therefore negate the need for further processing to provide the desired particle size distribution. Where spray drying is used as the drying method, it is preferred that the dried powder is cooled as soon as possible after the drying step, for example by conveying it from the drying stage to the next stage (e.g. a storage stage) in a cooled, low humidity current of air.

The above range of processing parameters has been found to provide a superior activation process for aluminium chlorhydrate compositions according to the invention, providing antiperspirant actives of superior efficacy.

In accordance with a further aspect of the invention there is provided a topical antiperspirant or deodorant composition comprising an effective amount of an activated basic aluminium salt prepared in accordance with the process described above.

Compositions which utilise aluminium salts produced according to the invention may be any of the topically applied forms, including sticks, roll-on lotions, aerosols, creams and soft solids, and pump spray formulations. Topical compositions according to the invention are preferably anhydrous; that is, the composition vehicle (i.e. the components of the composition, excluding the antiperspirant active salt itself) contain less than about 2%, more preferably less than 1% by weight of water. It is also preferred that topical compositions deliver the antiperspirant active as a suspended solid, and not as a solution, though topical compositions containing antiperspirant active solutions are also contemplated.

Although not limited as such, the compositions formed according to the invention may have particular utility in propellant driven aerosol compositions, in which zirconium based actives, currently the most efficacious available, are prohibited in certain countries. Topical compositions containing actives formed according to the invention may be formulated using those cosmetic ingredients which are used in the formulation of the particular topical composition, depending on the product form. Formulation of salts produced according to the invention may readily be carried out by those skilled in the art.

Conveniently, actives formed by the process according to the invention have a relatively high proportion of polymers contained in Band III compared to those in Band II of the Standard Basic Aluminium Chloride Solution Size Exclusion Chromatogram of the Size Exclusion Chromatography Test, as described in U.S. Pat. No. 4,359,456, the content of which is incorporated herein by reference. Preferably, the ratio of Band III to Band II material is greater than about 3:1. Conveniently the level of Band III material is more than about 55% by weight of the aluminium polymer species and it is preferred that this level is greater than 70% by weight. Conveniently, the amount of Band II material is less than about 25% by weight of the polymer species, with a level of less than 20% by weight being particularly preferred.

Characterisation of materials containing species differing in size by means of size exclusion chromatography (SEC) is generally known. Two size exclusion chromatographic procedures are required for the complete characterisation of the basic aluminium compounds of this invention. Method 1 permits the characterisation of materials on the basis of the percentage of aluminium in species greater than 100 Angstroms in size. Method 2 leads to a characterisation on the basis of the percentage of aluminium in species less than 100 Angstroms in size. The two methods will now be described.

Chromatographic Method 1

For the determination of the percentage of aluminium in polymeric species having a size greater than 100 Angstroms (i.e. Band 0 material), a 30 cm by 7.5 mm internal diameter stainless steel column was used. This was packed with a porous silica, available commercially as Porasil from Waters Corporation. The silica was characterised as having a particle size range of 37 to 55 micrometers, an average pore size of 125 Angstroms, a pore volume of 1.0 cc/g and a surface area of 320 m²/g.

The column was packed using a dry packing technique together with lateral tapping as described in “Silica Gel and Bonded Phases, Their Production, Properties and Use in LC”, by R P W Scott, Published by John Wiley and Sons, 1993, page 58.

Following packing, the eluent, consisting of an aqueous solution of 0.1 molar sodium nitrate and 0.01 molar nitric acid in deionized water, was first introduced from the bottom of the column at a flow rate of 1.0 ml/minute and passage continued until the exiting eluent was free from air bubbles. The eluent flow was then rearranged to feed to the top of the column and the column incorporated into a system comprising the sequence: sample loop injector, column, and refractive index detector (e.g. Waters R410). The detector was linked to an integrator that was used to monitor the separated fractions as they were eluted from the column. A standard eluent flow rate of 1.0 ml/minute was established.

In order that the column may be conditioned effectively and also to provide a standard material to qualify the performance of the column, a standard basic aluminium chloride was required. This was prepared by taking a sample of a 50% by weight aluminium chlorhydrate solution, available commercially as Aloxicoll-L from BK Giulini Chemie GmbH and Company OHG and characterised as having an aluminium to chlorine molar ratio of 2.01. This was diluted with deionized water to provide a 10.0% by weight aluminium chlorhydrate solution, and the solution heated in a closed vessel at 100° C. for 42 hours. The solution was spray dried to give the Standard Basic Aluminium Chloride Powder. The column was conditioned by injecting successive 500 microlitre samples, prepared from the Standard Basic Aluminium Chloride Powder and deionized water to contain 1.25% by weight aluminium, until a constant chromatogram was achieved.

A 200 microlitre sample, prepared from the Standard Basic Aluminium Chloride Powder and deionized water to contain 1.25% by weight aluminium, was then injected and two fractions corresponding to the bands in the chromatogram were collected and analysed for aluminium by plasma emission spectroscopy. The percentage of the total aluminium which appeared in the fraction eluted at the void volume (sometimes called the exclusion volume) of the column was 13.0% by weight and was considered as that deriving from polymeric material of a size greater than 100 Angstroms in effective diameter. Complete elution of all the aluminium in a sample applied to the column was checked by direct analysis of another sample of the same volume.

To prepare test solutions of materials for analysis for their Band 0 content, those already in solution were used undiluted unless the aluminium concentration exceeded 1.25% by weight, in which case they were diluted with deionized water to provide a solution containing 1.25% by weight aluminium. Solid materials (e.g. spray dried powders) were dissolved in deionized water to give solutions containing 1.25% by weight aluminium. These solutions were treated in an ultrasonic bath (e.g. Camlab Transsonic T660/H) for 2 minutes before application to the column.

Chromatographic Method 2

The analytical procedure used to determine the percentage of aluminium in species having a size less than 100 Angstroms (i.e. material in Bands I, II, III, and IV) was performed using a stainless steel column of dimensions 30 cm long and 7.0 mm internal diameter. This was packed with spherical porous silica of nominal particle size 5 micrometers diameter, an average pore size of 50 Angstroms diameter, a pore volume of 0.8 cc/g and a surface area of 450 m²/g. A suitable silica was that available commercially as Nucleosil 50 from Macherey-Nagel GmbH.

Although the columns used in the actual method employed by the Applicants were obtained ready packed from Jones Chromatography Limited of Hengoed, Mid-Glamorgan, Wales, if it were necessary to pack a column with the silica it could conveniently be carried out by the high-pressure slurry method (see “Silica Gel and Bonded Phases, Their Production, Properties and Use in LC”, by R P W Scott, Published by John Wiley and Sons, 1993, page 60) using hexane as the packing medium. In all cases the column would be equipped at the bottom with a zero dead volume fitting containing a 2 micrometer porosity stainless steel support and after packing would be capped with another zero dead volume fitting containing a 2 micrometer stainless steel frit.

The packed column was connected into a chromatographic system consisting of an automatic sampler, high-pressure pump, column, and a differential refractive index detector to monitor sample fractions as they were eluted. The refractive index detector was linked to an integrator to provide a real-time chromatogram and a data system that was programmed to calculate the relative chromatographic band areas of the fractions as a function of their elution times. The system was instructed to measure the areas of bands not resolved to the baseline by dropping perpendiculars from the lowest point of the valleys separating the bands to the baseline.

Newly packed columns were eluted with 200 ml of methanol at a flow rate of about 10 ml/minute, using a high pressure pump, to consolidate the bed and wash out the packing medium. This was followed by a change of eluent to the medium to be used for the analytical separations, in this case an aqueous solution containing 0.1 molar sodium nitrate and 0.01 molar nitric acid, and elution continued at a rate of 0.5 ml/minute until a flat base-line was achieved.

To provide a sample for conditioning the column and to act as a calibration standard a Standard Basic Aluminium Chloride Solution was prepared. This was carried out by dissolving 52.1 g of aluminium powder (99.97% aluminium by weight, grade 20/D supplied by The Aluminium Powder Company Limited of Holyhead, Anglesey, North Wales) in a solution of 93.2 g of aluminium chloride hexahydrate (supplied by SigmaAldrich Company Limited of Gillingham, Dorset SP8 4XT, UK) in 354.7 g of deionized water at about 90° C. in a stirred vessel equipped with a reflux condenser. When all of the aluminium had dissolved the solution was filtered to remove traces of insoluble impurities and allowed to cool to room temperature. This gave a Standard Basic Aluminium Chloride Solution that contained 12.5% aluminium by weight and 0% as polymers greater than 100 Angstroms in effective diameter (i.e. Band 0).

The column was conditioned by the application of multiple injections of 10 microlitre samples of the Standard Basic Aluminium Chloride Solution, diluted to 2.5% aluminium by weight, until a constant chromatogram was obtained from successive injections.

To prepare test solutions of materials for analysis for their Band I, II, III, and IV contents, those already in solution were used undiluted unless the aluminium concentration exceeded 2.5% by weight aluminium, in which case they were diluted with deionized water to provide a solution containing 2.5% by weight aluminium. Solid materials were dissolved in deionized water to give solutions containing 2.5% by weight aluminium. These solutions were treated in an ultrasonic bath for two minutes then filtered through 0.2 micrometer porosity cellulose acetate filter units. The preparation of the test solutions was carried out within 10 minutes of their application to the column. Sample solutions were applied to the top of the column as 1 microlitre injections and eluted at a rate of 0.5 ml/minute.

When a sample of Standard Basic Aluminium Chloride Solution was diluted to 2.5% aluminium by weight and applied to the column four main bands were obtained. They were characterised by means of the ratio of the retention times of the principal peak in each band to the retention time of the peak due to the totally included species (in the case of basic aluminium chlorides the totally included species arise from the presence of hydrochloric acid. This can be shown by comparison of its retention time with that of a sample of 0.01 molar hydrochloric acid.) and their chromatographic band areas expressed as percentages of the total chromatographic band area representing aluminium-containing material:

| | Band I | Band II | Band III | Band IV |
|---|---|---|---|---|
| Relative | 0.66 | 0.75 | 0.81 | 0.94 |
| retention time | | | | |
| (minutes) | | | | |
| Band area (% of | 26.1 | 61.3 | 8.4 | 4.2 |
| total aluminium | | | | |
| band area) | | | | |

Comparison of the total aluminium content of the eluted fractions representing Bands I to IV with that of another sample of the same volume that had not passed through the column showed that there was complete elution of aluminium species from the column. In a further experiment it was found that the relative aluminium contents of the separated fractions, expressed as percentages of the total aluminium contents of Bands I to IV, agreed closely with the relative area percents determined by integration of the signals from the refractive index detector for the same bands.

It will be appreciated by those skilled in the art that mechanisms of separation other than the principal mechanism of size exclusion may play a part in this type of chromatography. Examples of the processes would be adsorption effects and hydrodynamic effects. Thus although it is possible for a given column and constant operating conditions to lead to invariable relative retention times, minor variations in particle size range and pore size distribution of the packing materials may lead to slight differences in relative retention times and the splitting of the main bands. In our experience with standard columns packed with different batches of the specified packing material, the four aluminium-containing bands consistently fall within the ranges indicated:

| | Band I | Band II | Band III | Band IV |
|---|---|---|---|---|
| Relative retention | 0.56-0.72 | 0.73-0.79 | 0.80-0.87 | 0.88-0.98 |
| (minutes) | | | | |

Quantitatively, the amount of aluminium in the various Bands expressed as a percentage of the total aluminium of the compound under test is given by:

% Aluminium, Band 0=

The percentage of aluminium in the fraction eluting at the column void volume according to Chromatographic Method 1

% Aluminium, Bands I, II, III, or IV=

$$( 100 - A ) \times \frac{\begin{matrix}Area of band corresponding to Band I , II , \\ III , or IV fraction\end{matrix}}{\begin{matrix}Sum of the areas of the bands corresponding \\ to Bands I , II , III , and IV\end{matrix}}$$

where A is the percentage of the total aluminium which is contained in polymers greater than 100 Angstroms and is determined by Chromatographic Method 1.

Compositions according to the invention may also be characterised by the presence of certain spectroscopic peaks, as determined by ²⁷Al solution NMR spectroscopy.

In the method of the invention, the aluminium polymers formed may be analysed by NMR techniques to show the presence of, and to quantify, different polymer species, which have characteristic peaks in the ²⁷Al NMR spectrum. An example of these is the peak at 62.5 ppm downfield from the resonance of [Al(H₂O)₆]³⁺. This peak has been attributed to the presence of a tetrahedrally coordinated aluminium atom at the centre of the complex ion [Al₁₃O₄(OH)₂₄(H₂O)₁₂]⁷⁺ by Akitt et al. (J.C.S. Dalton Transactions 1972 p604), the structure of which was first established by G Johansson (Acta. Chem. Scand. 1960 Vol 14 p771). This ion has been subsequently referred to as the Al₁₃O₄₀ ion by Schonherr et al (Zeitschrift fur Anorganische und Allgemeine Chemie, 502, 113-122 (1983)). The desired level of this ion in the aluminium salts of the invention is detailed earlier in the specification.

A set of broader peaks which are detectable at between 64 and 76 ppm downfield from [Al(H₂O)₆]³⁺ correspond to the AlP₁, AlP₂, and AlP₃ polymer species referred to by Fu and Nazar in the above referenced paper. For the purposes of the analyses described herein, these peaks are grouped together and referred to as representing AlPₓ species. The desired level of these species represents at least 40%, in particular at least 47%, and especially at least 54% by weight of the aluminium present.

For the quantitative determination of the percentage attributable to the various peaks, it is recommended that an external calibration standard having a resonance position outside the range of the spectrum under investigation be used. A suitable standard and method of use is aqueoussodium aluminate solution (concentration 0.1M, resonance position δ=80 ppm), contained in a sealed 5 mm NMR tube held concentrically inside a 10 mm NMR tube; the annular space between the two tubes being filled with analyte solution, and the aluminate standard being freshly made up and calibrated for each series of experiments. This latter calibration can be performed using an aqueous solution containing a known concentration (eg 0.02M) of an aluminium salt, such as aluminium nitrate (resonance position δ=0 ppm), as a primary standard. In the calibration procedure, the primary standard is placed in the annular space between the two NMR tubes. From the ²⁷Al NMR spectrum of this system, the effective concentration of aluminium in the tube containing the external standard is calculated according to the equation:

M_{S}=[I_{S}/I_{A}]×M_{A}

where

M_{S} is the effective molar concentration of aluminium in the external standard solution;

M_{A} is the molar concentration of aluminium in the primary standard solution;

Iₛ is the area of the peak corresponding to the external standard (at δ=80 ppm for sodium aluminate); and

I_{A} is the area of the peak corresponding to the primary standard (at δ=0 ppm for aluminium nitrate).

Thus Ms is the ‘calibration factor’ of the sealed tube of the external standard, and the use of this tube, as indicated above, with subsequent analyte solutions of unknown composition allows the amount of aluminium associated with particular peaks in the spectrum resulting from the analyte solution to be quantified.

In our experiments, all NMR measurements were carried out at room temperature using a Bruker Avance DRX 500 spectrometer with a probe free from the background aluminium signal. Sample tubes were made from quartz (also free from background aluminium signal). The aluminium concentration of the analyte solutions whose polymer species were to be determined was in the range 0.3M to 1.0M. Spectra were obtained within 10 minutes of preparing the analyte solutions. The concentration of Al₁₃O₄₀ ions present was quantified using the area of their peak at 62.5 ppm, together with an appropriate scale factor. The concentration of AlPₓ ions present was quantified using the area of their peaks between 64 and 76 ppm, together with the scale factor used for the Al₁₃O₄₀ ion.

The invention will now be further illustrated by way of the following non-limiting examples.

### EXAMPLE 1

AlCl₃₀.6H₂O (99% purity, ex. Aldrich) was dissolved in 250 g distilled water in a round bottomed flask, and aluminium foil (99.8% purity, ex. Aldrich) or aluminium powder (99.97% purity, ex. Alpoco) was added and a reflux condenser was fitted. The exact amounts of aluminium chloride and aluminium required were calculated such that the required ratios of Al/Cl would be present in the resulting solution (in this example, for all samples produced, Al:Cl=1.8:1.0), and also such that the aluminium concentration in the resulting solution would be that required. The mixture was stirred and heated at 100° C. for 42 hours. As the heating commenced, the aluminium started to dissolve, causing evolution of hydrogen gas which was slow at first, and became more vigorous as the temperature was raised. Most of the aluminium had dissolved (and hence hydrogen evolution had ceased) after about 5 hours. In this example, the aluminium dissolution step and the ageing step were carried out sequentially, at the same temperature (i.e. 100° C.).

After the heating steps, the solution was cooled, filtered and freeze dried to provide a white/pale yellow powder. Samples were analysed by SEC and ²⁷Al NMR, as described above.

Results

| | | |
|---|---|---|
| Final Al | SEC Bands (%) | ²⁷Al NMR |

| conc. | | Band | Band | Band | | AlP_{X} |
|---|---|---|---|---|---|---|
| (wt %) | Band 0 | II | III | IV | Al₁₃ | (%) |
| 1.00 | 15.5 | 17.4 | 54.8 | 12.3 | 1.7 | 40.5 |
| 1.25 | 10.7 | 21.8 | 59.9 | 7.7 | 1.8 | 46.7 |
| 1.50 | 8.2 | 21.8 | 63.7 | 6.3 | 2.1 | 49.9 |
| 1.75 | 2.9 | 18.2 | 71.5 | 7.5 | 2.8 | 57.4 |
| 2.00 | 0.7 | 19.8 | 73.0 | 6.6 | 3.5 | 55.7 |
| 2.50 | 0.2 | 25.5 | 70.1 | 4.3 | 3.4 | 54.0 |

The results indicate that, based on the amount of Band III material and also the amount of AlPₓ species present, that the preferred aluminium concentration at an ageing temperature of 100° C. is between 1.75 and 2.0%.

### EXAMPLE 2

In a related example, the method as described above was used to generate solutions which had final aluminium concentrations of 0.85%, 1.75%, 2.63% and 3.5% by weight aluminium, and were aged at 100° C. for 24 hours. The solutions were freeze dried, and the resulting materials were ball-milled and sieved to pass a 75 μm screen. The resulting powders were formulated into topical roll on lotion compositions containing 22% by weight active salt, 3% Bentone 38, 1% ethanol, 1% propylene carbonate, and 73% DC345 volatile silicone. The compositions were then tested in a hotroom to determine their antiperspirant efficacy, and sweat rate reduction results were obtained for each composition. The method use for evaluating efficacy was that described in U.S. Pat. No. 4,359,456, Test method II (col. 11), except that the panel consisted of at least 30 women who had not used antiperspirant for 17 days before the test; also, in terms of the analysis of data, the % reduction was not calculated for each day separately, and significance was not calculated by applying Duncans Multiple Range Test.

Results

| Al. concentration (%) | Sweat Rate Reduction (%) |
|---|---|
| 0.85 | 38.5 |
| 1.75 | 44.5 |
| 2.63 | 39.4 |
| 3.5 | 37.3 |

These results indicate that the preferred aluminium concentration in the final solution is at or close to 1.75%.

### EXAMPLE 3

A sample was prepared in a manner similar to that described in Example 1, except that the sample was heated at a temperature of 100° C. for 24 hours, and had an aluminium concentration in the final solution of 1.75%. The resulting solution was then spray dried, formulated into a topical roll on composition and applied to subjects who subsequently were examined for the sweat rate reduction in a hotroom. The test was conducted against commercially available samples of aluminium chlorhydrate and activated aluminium chlorhydrate.

The salt prepared according to the method of the invention had a sweat rate reduction when measured in the hotroom of 43.2%, compared to aluminium chlorhydrate, which had a sweat rate reduction of 24.5%, and activated aluminium chlorhydrate, which had a sweat rate reduction of 32.0%.

### EXAMPLE 4

Example 4 illustrates the significance of water content in the dried salts made from solutions prepared according to the invention.

Solutions were prepared according to the general method described in relation to Example 1 above. The samples were aged at a temperature of 100° C. for 24 hours, at an aluminium:chlorine ratio of 1.8:1.0, and a final aluminium concentrations of between 0.875 and 1.75 wt %. Thereafter, the salts were freeze dried to the variety of water contents quoted.

Each of the salts was formulated into a topical roll on lotion formulation as detailed in Example 2. The sweat rate reduction performance of the formulations was assessed according to the procedure given in Example 2. A further assessment according to this procedure was also made after the formulations had been stored in sealed containers for 6 months at 20° C.

Results

| Final Al | | | |
|---|---|---|---|
| conc. in aged | Water | Sample | Sweat rate |
| soln. (wt %) | content (%) | stored? | Reduction (%) |
| 1.75 | 14.8 | No | 43.4 |
| | | Yes | 31.2 |
| 0.875 | 12.4 | No | 36.2 |
| | | Yes | 19.6 |
| 1.75 | 11.6 | No | 41.4 |
| | | yes | 42.6 |
| 1.75 | 10.3 | No | 45.8 |
| | | Yes | 45.3 |

The results indicate that the salts produced according to the method of the invention are sensitive to water content, with a water content of greater than 12 wt % leading to considerable deterioration in the efficacy of the salt in a topical composition after a period of time in storage.

### EXAMPLE 5

Example 5 further illustrates the stability of low water content antiperspirant salts prepared according to the method of the invention.

A solution was prepared according to the general method described in relation to Example 1, using an aluminium concentration of 1.75% by weight, HCl instead of AlCl₃ at a level sufficient to give an Al/Cl ratio of 1.8:1.0, and a total time of 24 hours at 100° C. Thereafter, the salt was sprayed dried to give a water content of 9.5% by weight. The resulting powder was stored at room temperature and its stability monitored using SEC and ²⁷Al NMR.

Results

| | SEC Bands (%) | | | | ²⁷Al NMR |
|---|---|---|---|---|---|
| Time (days) | Band 0 | Band II | Band III | Band IV | AlPₓ (%) |
| 0 | 0.1 | 16.6 | 74.3 | 9.1 | 48.6 |
| 50 | | | | | 48.2 |
| 161 | 0.1 | 16.5 | 74.8 | 8.6 | |

These results indicate remarkably good stability for this low water content aluminium salt.

### EXAMPLE 6

An antiperspirant active was prepared according to the general method described in Example 1, except that the initial dissolution step was carried out at an aluminium concentration of 10% by weight of aluminium, and a solution of HCl was used rather than AlCl₃. The dissolution step was carried out at a temperature of 90° C., and at an aluminium:chlorine ratio of 1.8:1. This solution was diluted once the aluminium was fully dissolved to provide a solution for ageing which had an aluminium concentration of 2 wt %. The ageing was carried out for a period of 28 hours at a temperature of 100° C., after which time the active was isolated by spray drying.

The resultant salt was incorporated into an antiperspirant roll on composition as described in relation to Example 2. When tested in a hotroom, the composition had a sweat rate reduction of 39.1%, as compared to a sweat rate reduction of 32% for conventional commercially available activated aluminium chlorhydrate.

### EXAMPLE 7

An antiperspirant active salt was prepared in a similar manner to that described in Example 6, except that a solution of AlCl₃ was used rather than HCl. Again a solution which contained 10 wt % aluminium was diluted to one which contained 2 wt % for the ageing step. The ageing step was carried out for a period of 24 hours at a temperature of 100° C. The resulting salt was spray dried and incorporated into a roll on composition, as described in example 2. The resultant composition showed a sweat rate reduction in hot room tests of 38.2%, compared to a sweat rate reduction of 32% for a commercially available activated aluminium chlorhydrate.

### EXAMPLE 8

Example 8 illustrates the benefit of sequential use of a high temperature followed by a lower temperature in the ageing step of the method of the invention.

An antiperspirant active was prepared according to the general method described in Example 1, except that aluminium powder, hydrochloric acid (s.g. 1.16), and distilled water were used in the initial dissolution step to give an Al/Cl ratio of 1.8:1.0 and an aluminium concentration of 12.5% by weight. The reaction was carried out at a temperature of 90° C. After the aluminium had fully dissolved, the solution was diluted to an aluminium concentration of 2.5% by weight of aluminium and aged at 120° C. for 1.5 hours in a sealed vessel. Following this treatment, the solution was diluted to an aluminium concentration of 1.75% by weight and the ageing step completed at 100° C. for 6 hours. The active aluminium salt was isolated by spray drying and analysed by SEC and ²⁷Al NMR.

Results

| SEC Bands (%) | | | | ²⁷Al NMR | |
|---|---|---|---|---|---|
| Band 0 | Band II | Band III | Band IV | Al₁₃ (%) | AlPₓ (%) |
| 0.1 | 13.8 | 80.3 | 5.8 | 6.2 | 55.2 |

This example illustrates that a desirable aluminium salt can be generated in a relatively short time by sequential use of a high temperature followed by a lower temperature in the ageing step. Particularly noticeable are the high levels of Band III material and AlPₓ material generated by this procedure.

### EXAMPLE 9

Example 9 further illustrates the benefit of sequential use of a high temperature followed by a lower temperature in the ageing step of the method of the invention.

An antiperspirant active was prepared according to the method described in Example 8, except that the first part of the ageing step, performed at 120° C. and 2.5% by weight aluminium, was continued for 2.5 hours, and the second part of the ageing step, performed at 1.75% aluminium, was continued for 1.5 hours at 98° C.

Results

| SEC Bands (%) | | | | ²⁷Al NMR | |
|---|---|---|---|---|---|
| Band 0 | Band II | Band III | Band IV | Al₁₃ (%) | AlPₓ (%) |
| 0.1 | 13.2 | 80.7 | 6.0 | 6.0 | 51.7 |

This example illustrates that a desirable aluminium salt can be generated in a very short time by sequential use of a high temperature followed by a lower temperature in the ageing step.

### EXAMPLE 10

Example 10 illustrates that the benefit of sequential use of a high temperature, followed by a lower temperature, in the ageing step, is found when the first stage of the ageing step is performed at the same aluminium concentration and the same temperature as the initial dissolution step.

An antiperspirant active was prepared according to the procedure described in Example 8, except that the primary dissolution and ageing steps were combined and carried out at a temperature of 100° C., over a period of 20 hours, and at an aluminium concentration of 1.75% by weight. At the end of this period, the temperature of the reaction mixture was lowered to 85° C. and heating continued for a further 30 hours at the same aluminium concentration. At the end of the ageing the active was isolated by freeze drying. Samples were analysed by SEC and ²⁷Al NMR as before.

| SEC Bands (%) | | | | ²⁷Al NMR | |
|---|---|---|---|---|---|
| Band 0 | Band II | Band III | Band IV | Al₁₃ (%) | AlPₓ (%) |
| 0.1 | 8.8 | 84.8 | 6.3 | 3.6 | 57.6 |

These results illustrate that an extremely simple process can be used to prepare a desirable aluminium salt.

### EXAMPLE 11

Antiperspirant salts prepared according to the invention may be incorporated into suspension aerosol products of the following composition using conventional processing methods.

| Component | Amount (wt %) | Function |
|---|---|---|
| Antiperspirant salt | 5.0 | Active |
| Volatile silicone¹ | 10.0 | Carrier/emollient |
| Talc | 1.0 | Tactile properties |
| Clay² | 0.75 | Suspending agent |
| Fragrance | 0.75 | |
| Hydrocarbon | to 100 | Propellant |

¹D5 (cyclopentasiloxane) grade, eg. DC 245 Fluid.
²Bentone 38V, ex Rheox.

1. D5 (cyclopentasiloxane) grade, eg. DC 245 Fluid.

2. Bentone 38V, ex Rheox.

### EXAMPLE 12

Antiperspirant salts prepared according to the invention may be incorporated into concentrated aerosol products of the following composition using conventional processing methods.

| Component | Amount (wt %) | Function |
|---|---|---|
| Antiperspirant salt | 21.0 | Active |
| Bentone gel VS-5 PC¹ | 18.5 | Suspension/dispersion |
| Volatile silicone² | 10.2 | Carrier/emollient |
| Dimethicone | 8.5 | Carrier/emollient |
| Isopropyl myristate | 1.5 | Emollient |
| Fragrance | 0.3 | |
| Isobutane | 40 | Propellant |

¹ex Rheox.
²D5 (cyclopentasiloxane) grade, eg. DC 245 Fluid.

1. ex Rheox.

2. D5 (cyclopentasiloxane) grade, eg. DC 245 Fluid.

### EXAMPLE 13

Antiperspirant salts prepared according to the invention may be incorporated into suspension antipersirant stick products of the following composition using conventional processing methods.

| Component | Amount (wt %) | Function |
|---|---|---|
| Volatile silicone¹ | 45.0 | Carrier/emollient |
| Antiperspirant salt | 20.0 | Active |
| Stearyl alcohol | 15.0 | Structurant |
| PPG-10 cetyl alcohol | 5.0 | Surfactant |
| Hydrogenated castor oil | 5.0 | Structurant |
| Phenyl trimethicone | 5.0 | Emollient |
| PPG-15 stearyl ether | 5.0 | Surfactant |

DC 345 Fluid, ex Dow Corning. Alternatively, DC 245 Fluid, ex Dow Corning, may be used.

1. DC 345 Fluid, ex Dow Corning. Alternatively, DC 245 Fluid, ex Dow Corning, may be used.

### EXAMPLE 14

Antiperspirant salts prepared according to the invention may be incorporated into soft solid/dry cream products of the following composition using conventional processing methods.

| Component | Amount (wt %) | Function |
|---|---|---|
| Volatile silicone¹ | 65.0 | Carrier/emollient |
| Antiperspirant salt | 24.0 | Active |
| C18-36 acid | 6.5 | Structurant |
| triglyceride/tribehenin | | |
| Dimethicone | 4 | Carrier/emollient |
| Fragrance | 0.5 | |

¹D5 (cyclopentasiloxane) grade, eg. DC 245 Fluid.

1. D5 (cyclopentasiloxane) grade, eg. DC 245 Fluid.

1. A method of providing an antiperspirant active having the empirical formula
Al₂(OH)₆₋ₐXₐ.YH₂O
wherein X is Cl, Br or I, Y is between 1.5 and 2.5 and a is 0.8 to 1.33, comprising heating an aqueous solution of aluminium halide or HX and aluminium metal for a time period sufficient for the aluminium metal to dissolve, and subsequently ageing the solution of dissolved aluminium for a period of 50 minutes to 21 days at a temperature of 80-130° C., such that the Al/X ratio in the resulting solution is between 1.5:1 and 2.0:1, and such that the aluminium concentration in the resulting solution is between 0.5 and 3.8 wt %.

2. A method according to claim 1, wherein the resulting material is dried to a water content of less than 12 wt %.

3. A method according to claim 1 or claim 2, wherein a is between 0.9 and 1.2.

4. A method according to any of claims 1-3, wherein the composition is heated in the aluminium dissolution step at a temperature of 90-100° C., preferably 95-100° C.

5. A method according to any of the preceding claims, wherein the ageing step is carried out for a period of 2 to 10 hours.

6. A method according to any of the preceding claims, wherein the ageing step is carried out at 110-130° C. for 0.5 to 10 hours and subsequently at 85-100° C. for 3 to 48 hours.

7. A method according to any of the preceding claims, wherein the aluminium concentration in the resulting solution is in the region 1.4-2.7% by weight.

8. A method according to any of the preceding claims, wherein the amount of Band 0 polymers in the resulting solution is 0.5-5% by weight.

9. A method according to any of the preceding claims, wherein X is Cl.

10. A method according to any of the preceding claims, wherein the ratio of Al:X in the final product is between 1.75:1 and 1.85:1.

11. A method according to any of the preceding claims, wherein the water content of the dried salt is in the region of 8-12%, preferably 8-10%.

12. A method according to any of the preceding claims, wherein the aged solution is spray dried.

13. A method according to any of the preceding claims, wherein the resulting antiperspirant active salt has a ratio of Band III to Band II in the Size Exclusion Chromatography Test of greater than 3:1.

14. A method according to any of the preceding claims, wherein the resulting antiperspirant active salt has a concentration of Al₁₃ species of less than 7% by weight, as determined by ²⁷Al NMR techniques.

15. A method according to any of the preceding claims, wherein the resulting antiperspirant active salt has a concentration of AlPₓ species of at least 40% by weight, as determined by ²⁷Al NMR techniques.

16. A method according to any of the preceding claims, wherein the dissolution step is carried out at substantially the same aluminium concentration as the ageing step.

17. A method according to any of the preceding claims, wherein the method does not involve a depolymerisation step.

18. An antiperspirant active salt comprising a polymeric aluminium salt made according to any of the preceding claims.

19. A topical antiperspirant composition for application to the human skin comprising an antiperspirant active according to claim 18.

20. A topical antiperspirant composition according to claim 19, wherein the composition vehicle comprises less than 1% water.
